(19) United States
(12) Patent Application Publication
(10) Pub. No.: US 2021/0059735 A1
(43) Pub. Date: Mar. 4, 2021

(54) CANNULATED LOCKING AND FIXATION SET FOR ORTHOPEDIC SURGERY

(71) Applicant: Martin RUSSI, Montevideo (UY)

(72) Inventor: Martin RUSSI, Montevideo (UY)

(21) Appl. No.: 17/006,098

(22) Filed: Aug. 28, 2020

(30) Foreign Application Priority Data
Aug. 30, 2019 (UY) 38.357

(51) Int. Cl. A61B 17/88; A61B 17/84

(52) U.S. Cl. CPC A61B 17/8861; A61B 17/848; A61B 17/846; A61B 17/842

(57) ABSTRACT

A cannulated locking and fixation set for orthopedic surgery, which allows to easily and simply lock useful elements for orthopedic surgeries or other surgeries that currently demonstrate a more complicated fixation system or not at all.

### FIELD OF THE INVENTION

The present invention refers to devices, arrangements or means used in surgeries, most preferably to maintain stabilized bone fractures or osteotomies in elongations or angular corrections during orthopedic surgeries. More particularly, the invention refers to a cannulated locking and fixation set that allows for double locking of the elements that pass through its interior for the subsequent fixation of the bone structures to be intervened, letting the process of locking and fixation be more practical, fast and efficient. Despite the present description makes reference to a cannulated locking and fixation set for orthopedic surgeries, it is clear that the invention may be considered and used without any inconvenience in any type of surgery wherein a body part must be secured.

### DESCRIPTION OF THE ART

Bone elongation, or surgical elongation, is a surgical process used to correct congenital bone malformations, post traumatic fractures or for patients who want to increase the length of their limbs. An osteotomy is used to fracture a bone in two parts, slightly separating the ends, allowing a new bone to form in the space. When the desired or possible length is reached, a healing phase is undertaken in which the bone is allowed to continue to heal. The procedure has the benefit of simultaneously increasing the length of the bone and the volume of the surrounding tissues.

In order to perform these surgical tasks, a fixator or external element is required to allow the surgery to be performed. Sometimes this external fixation system can generate intolerance due to pain, irritation, and infection in its trajectories that make bone lengthening difficult, when it crosses soft tissues such as fascia muscle and tissues peripheral to the bone structure. There are systems of internal bone lengthening, (magnetic or electric telescopic nails) that improve the tolerance of soft parts during lengthening, but also present shortcomings and limitations. These are requiring an adequate size of bone length or channel size for placement, sacrificing joints for placement, or crossing the physis in a growing skeleton.

Acute deformity corrections may require plate and screw osteosynthesis as treatment for acute fractures. Currently, extra-medullary internal bone fixation systems (osteosynthesis plates) for orthopedic surgery can use locking screws and even with a variable angle by determining a screw to plate connection in order to achieve a more stable and rigid fixation plate-bone. Accordingly, injury to the periosteum and bone necrosis in that area by compression is avoided, as unlocked plates do, thus behaving like an internal fixator.

Another known type of fixator is the Ilizarov, which is a compression-distraction device that consists of a series of rings on which bone fixation elements, such as transosseous Kirschner wires-pins, are fixated, crossed in pairs and placed under tension. The wires-pins cross the muscles and soft tissue, avoiding vascular nerve structures, according to well-known lines of direction, thus creating a fixation system that is both stable and elastic. Through the wires, axial corrections, compression, distraction, displacement of fragments at a distance, etc., can then be applied thereon. The rings, in turn, are connected to each other by means of connecting elements such as bars, plates and/or hinges.

Both the osteosynthesis plate and the Ilizarov rings have proven to work well in practice. However, we will focus on the drawbacks in the fixation or locking of the bone fixation elements in both the plate and the rings. As it is known in the art, bone fixation elements such as Kirschner pins or cerclage wires or cables do not currently show a specific form of fixation to the osteosynthesis plates. The way of fixating to the Ilizarov rings is through a very cumbersome nut and locknut set. Professional surgeons often want to tighten the bone fixation element before fixating it to the plate, but currently, they do not have a mechanism for doing so and tightening the rings involves a complicated task, given the bone fixation element must be tightened and at the same time trying to fixate the nut. When placing the nut, extra care must be taken to keep the bone fixator tight so the locknut can be mounted—then fixated and the correct tension provided. If the connection to the ring is not tightened correctly, instabilities can occur which can lead to complications during surgery. Practice has shown that fixating the nut and locknut is far too inconvenient for professionals, who must require great experience and patience in order to perform said procedures properly.

In light of the above, it would be highly convenient to provide a novel arrangement or set that would allow for the fixation and locking of the bone fixation elements in the plates and rings in a much more practical, quicker and easier way, avoiding the above disadvantages.

### BRIEF DESCRIPTION OF THE INVENTION

It is therefore an object of the present invention to provide a new locking and fixation set for orthopedic surgeries, which provides greater ease, practicality and speed in carrying out the fixation and locking of bone fixation elements.

It is also an object of the present invention to provide a fixation and locking set that provides for double locking of the bone fixation elements.

It is also an object of the present invention to provide a cannulated fixation and locking set which allows for the generation of locked Kirschners for osteosynthesis or tension locked Kirschners, a fundamental principle for the development of bone fixation systems with the concept of Ilizarov (tension Kirschner), which can be incorporated to the systems of locked plates, determining the concept of Ilizarov but in an internal fixation, having the advantages that the wires or pins of Kirschner to tension do not cross soft parts like muscle, fascia and skin, thus avoiding the complications of infection in the path of the pin, as well as irritation and pain intolerance.

It is yet another object of the present invention to provide a cannulated fixation and locking set that can generate mixed osteosynthesis systems, i.e., systems of internal endomedullary and external extramedullary osteosynthesis, by being able to lock endomedullary interlocking elements associated with a plate.

It is furthermore another object of the present invention to provide a cannulated locking and fixation set presenting a threaded cannulated fixation element through which the respective bone fixation element passes and is retained permanently.

It is yet another object of the present invention to provide a cannulated locking and fixation set for orthopedic surgery comprising at least one internal fixation element with an external threaded section of conical development, with an upper and lower face interconnected by an internal through channel, with at least one cross section provided in said threaded section up to said lower face, and at least one external fixation element featuring at least one conical hole with at least one threaded part receiving said internal fixation element.

### BRIEF DESCRIPTION OF THE DRAWINGS

For greater clarity and understanding of the object of the present invention, it has been illustrated in several figures, in which it has been represented in one of the preferred embodiments, all by way of example, wherein:

FIGS. 1 and 2 are seen in perspective of a portion of the cannulated locking and fixation set, in accordance with a preferred first embodiment of the invention;

FIG. 3 is a side view of a portion of the locking set in FIG. 1, where an internal fixation element and a portion of an external fixation element can be seen;

FIG. 4 is a side view of a portion of the external fixation element in FIG. 3;

FIG. 5 is a front view of an external fixation element in accordance with a preferred first embodiment of the invention;

FIGS. 6 to 9 show different views of the internal fixation element according to a preferred first embodiment;

FIG. 10 is a perspective view of the internal fixation element in accordance with a second preferred embodiment;

FIG. 11 is a partial view of a femur fracture in which an external fixation element is arranged in accordance with a second preferred embodiment of the invention, wherein the use of a fixation sphere and a Cerclage cable can be observed;

FIGS. 12 and 13 show different views of an external fixation element, in accordance with a third preferred embodiment of the invention, wherein the use of an Ilizarov ring and a Kirschner wire can be observed;

FIGS. 14 to 16 show different views of an external fixation element, in accordance with a fourth preferred embodiment of the invention, where a fixation thimble and a Kirschner wire can be seen; and

FIG. 17 shows a partial view of a bone structure to be treated, in which the external fixation element of the fourth embodiment is applied according to FIGS. 14 to 16, wherein the use of the thimble in conjunction with an osteosynthesis plate can be observed.

### DETAILED DESCRIPTION OF THE INVENTION

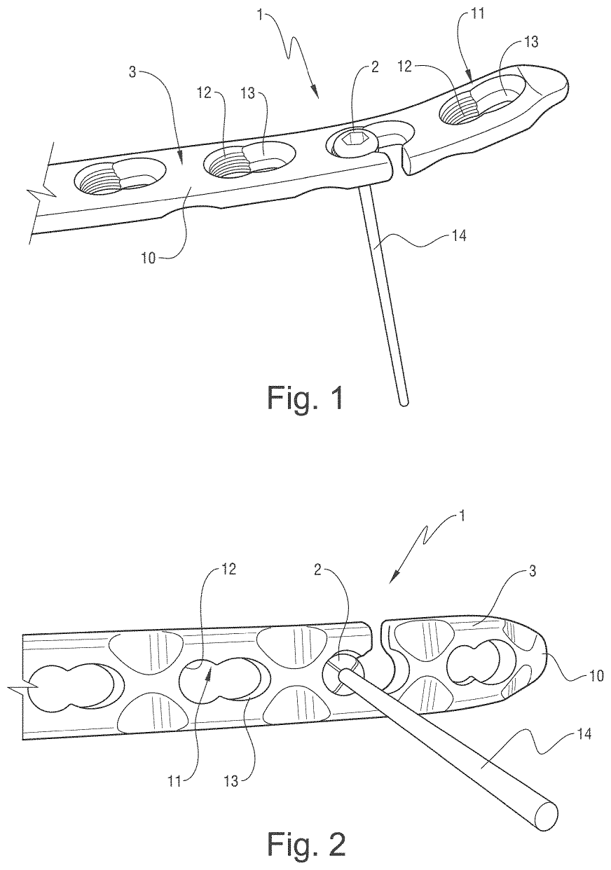

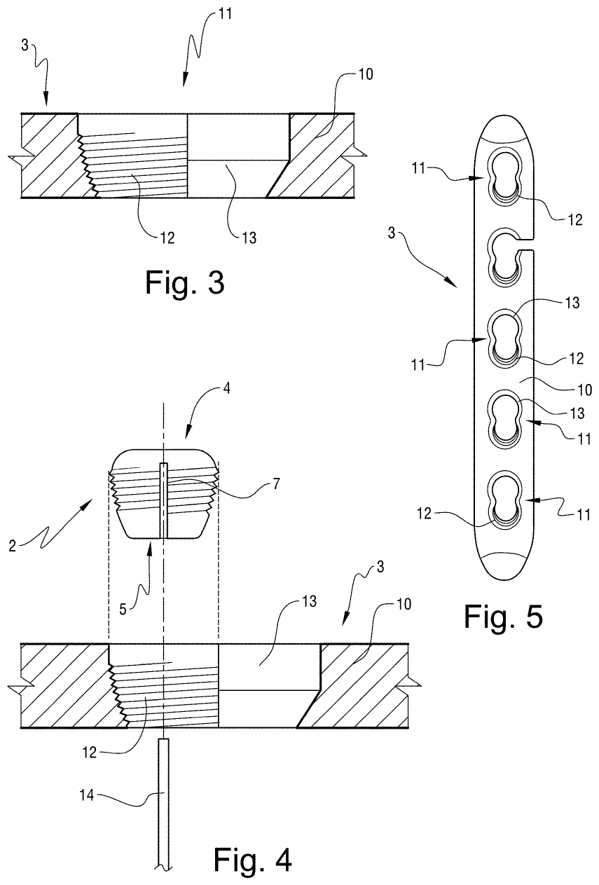

Referring to the figures, it is noticed that the invention consists of a novel cannulated locking and fixation set, which provides a double-locking arrangement that allows the fixation and locking of bone fixation elements in a much more practical, quick and easy way. According to FIGS. 1 to 9, the set of the invention is indicated by the general reference 1 and comprises at least one internal fixation element 2 and at least one external fixation element 3.

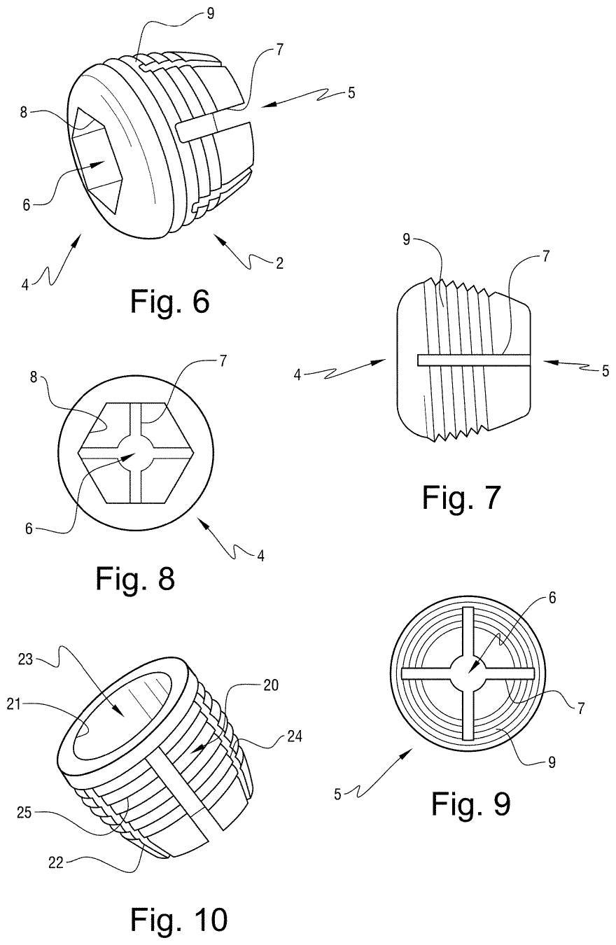

The internal fixation element 2 preferably comprises a revolving part with at least a threaded conical section as referred to below, with an end or upper side 4 and an end or lower side 5 interconnected by an internal through channel 6 or cannulated, and with a cross-section 7 in that lower side 5 extending towards the upper side 4 but not reaching that end or upper side, as best illustrated in FIGS. 6 to 9. Upper side 4 is machined to fit an adjustment tool (not shown), and may be of any shape to allow for the fitting of the adjustment tool such as a hollow socket wrench or the like. Also, the internal fixation element 2 has at least a tapered external threaded section 9 which will allow threaded mounting on the external fixation element 3 as described below.

The external fixation element 3, in a first preferred embodiment, FIGS. 1 to 5, comprises an osteosynthesis plate 10 which has a plurality of tapered holes 11, each of which has at least one threaded part 12 which receives the internal fixation element 2, and a smooth part 13. Thus, the internal fixation element 2 is threaded into the external fixation element 3 through the tapered threaded hole 12, with threaded adjustment that can be made with the adjustment tool. As the internal fixation element 2 is threaded into the tapered threaded hole 12, it can be operated between open and closed positions by means of the adjustment tool. That is, since both the internal fixation element 2, which has the cut-out(s) 7, and the tapered threaded hole 12 are tapered, when the adjusting tool is rotated the internal fixation element 2 is “closed” through the cross cut, allowing a bone fixation element 14 to be griped, adjusted and secured as it passes through it.

In this way, the professional surgeon prior to surgery can prepare the osteosynthesis plate 10 with the internal fixation elements 2 already threaded into it to avoid closing the internal channel 6 completely, so the bone fixation element 14 can then pass through. Thus, at the time of surgery the professional only has to worry about tightening the bone fixation element well and adjusting the internal fixation element 2 through the wrench or adjustment tool. Although a Kirschner wire has been illustrated in FIGS. 1 to 3 as bone fixator 14, this does not imply that the invention is limited to it, but that other bone fixators can be considered and used without any inconvenience as seen below.

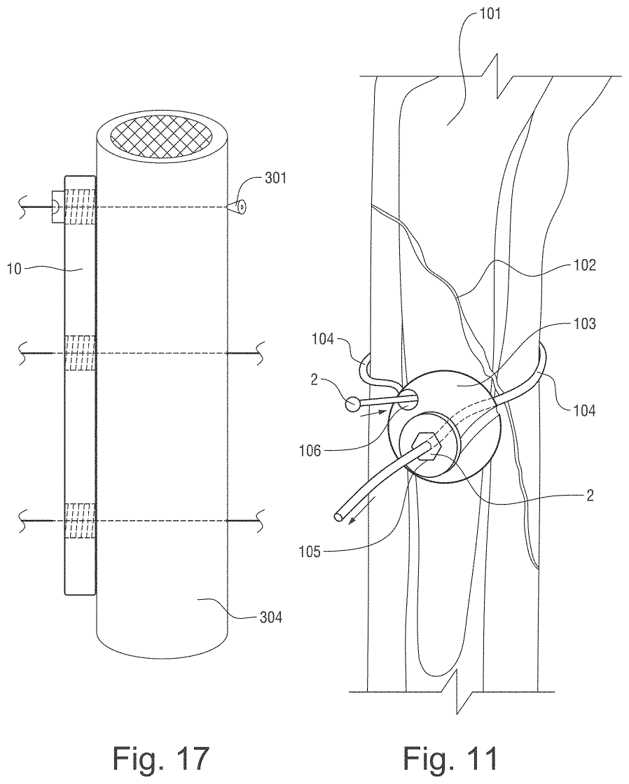

According to FIG. 10 and with an alternative of the invention, the internal fixation element may comprise a cup shape 20 which also has an upper side 21, a lower side 22, an internal channel 23, a cross cut 24 forming elastic sections for deformation and tightening of a long-formed piece, rope, etc., and an external threaded portion 25.

According to a second preferred embodiment, FIG. 11, a femur 101 can be observed, which presents a periprosthetic type femur fracture 102. For treatment, a fixation sphere 103 and a Cerclage cable 104 can be used as an external fixation element for bone. The fixation sphere 103 has at least one main threaded hole 105 in which an internal fixator 2 as described in FIG. 6 to 9 or 10 will be threaded in, and a secondary threaded hole 106 in which a second internal fixator 2 will be housed.

In this way, the cable from Cerclage 104 is passed through internal fixation element 2 in the main hole 105, “clamping” the fractured femur area, and finally passing it through internal fixation element 2 in the secondary hole 106. The cable 104 is then tightened and the internal fixation element 2 of the main hole 105 is adjusted. This provides a double locking of the fixation sphere 103 in a much more practical, quick and easy way, without the need for complications for the professional surgeon.

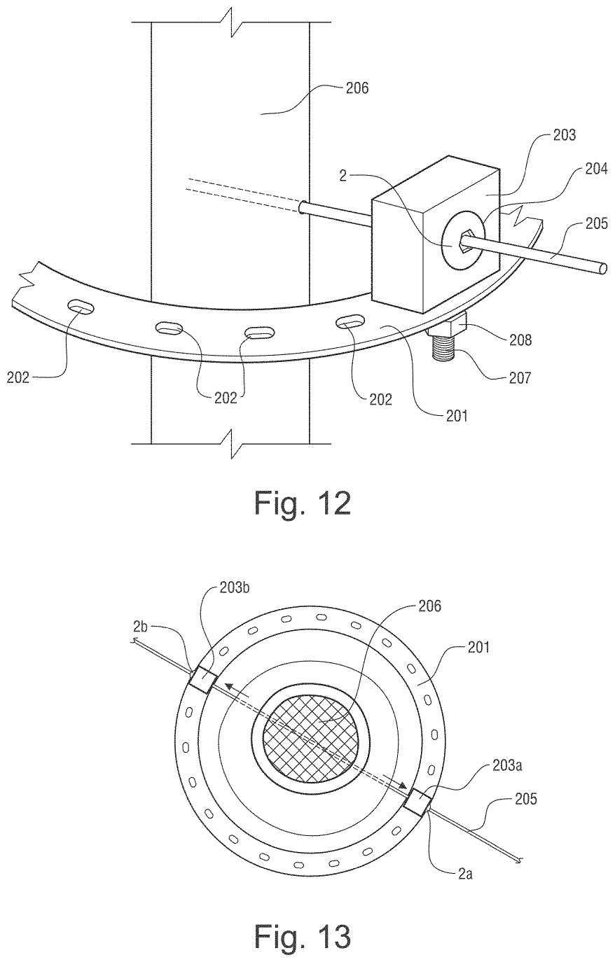

According to FIGS. 12 and 13, in a third preferred design, there is at least one Ilizarov ring 201 with a number of holes 202 in which a cubic additional fixation element 203 with a tapered threaded hole 204 is mounted, in which an internal fixation element 2 as described in FIG. 6 to 9 or 10 is threaded. In this case, a Kirschner wire 205 is used as a bone fixation element, which is arranged parallel to the ring face. In a form of application, but not limited to the invention, in order to carry out the fixation of a bone structure 206 using the wire 205, one must first fixate the set defined by the cubic complementary fixation element 203a and internal fixation element 2a, and then tighten the wire 205 and adjust the set formed by the cubic complementary fixation element 203b and internal fixation element 2b, thus providing a double locking that is much more practical, safe and easy.

It should be noted that the fixation process of Ilizarov's rings to each other is well known in the art and for that reason, no descriptive details about them will be given. Moreover, the complementary cubic fixation element 203 is mounted on the Ilizarov ring 201 by means of a set of pins 207 and nut 208.

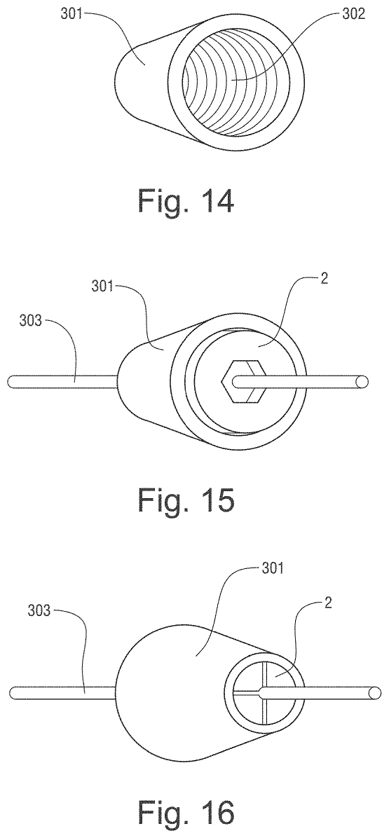

Referring to FIGS. 14 to 17, in a fourth preferred embodiment, a conical thimble 301 is presented as an external fixation element which has an internal thread 302 to allow the threaded mounting of an internal fixation element 2 as described in FIG. 6 to 9 or 10. This conical thimble 301 can be used in conjunction with an osteosynthesis plate 10 as described in FIGS. 1 to 5, as best illustrated in FIG. 17. The joint use of the thimble 301 is not limited to the plate, but can be complementary to any of the external fixation elements mentioned above. It can also be used with any type of bone fixation element such as a needle 303.

Therefore, according to FIG. 17, in order to carry out the fixation, the needle 303 is first passed through the osteosynthesis plate 10 and the bone structure 304 to be treated. Finally, the needle 303 is “brought in” and tightened sufficiently to fixate it in the osteosynthesis plate by means of the internal fixation element 2, which is already threaded in.

It should again be noted that, in all cases, adjustment of internal fixation element 2 is performed using a hollowed-out Allen wrench or cannulated screwdriver to allow tensioning of the fixation element to the bone prior to locking or any related adjustment tools. Thus, the present invention can be used in combinations of bone fixation elements which can be selected from the group consisting of Cerclage wires, pins or Kirschner wires, pins, or a combination thereof, as well as internal fixation elements which can be selected from the group consisting of osteosynthesis plates, or external fixation such as Ilizarov rings, fixation thimbles, fixation spheres, or a combination thereof.

In this way, the cannulated fixation and locking set is formed and constructed for orthopedic surgeries of the present invention, which allows to lock in an easy and simple way useful elements for orthopedic surgeries or other surgeries that nowadays present a more complicated fixation system or do not present it at all, due to the set formed by the internal conical, cannulated and threaded fixation element that is fixated in the external fixation element that presents a conical threaded hole for its reception. As the internal fixation element is tapered, cannulated and threaded with the cross cuts, the simple fact of adjusting it with a wrench allows the clamping of the bone fixation element in a much more practical, efficient and easy way.

In turn, by means of the invention, the double locking cannulated set of the invention can be added to the conventional systems of the prior art, generating the possibility of adding in these systems the incorporation of locked Kirschner or locked tension Kirschner to reduce multi-fragmentary fractures with small fragments even in joints that nowadays are very difficult to stabilize, and add the concept of tension elements that can allow an earlier support of the patient (concept of Ilizarov in its circular system of external fixation). The invention may also make it possible to incorporate into the system of locked plates the association in their holes of cerclages with wire, cable or thread very much used today in peri-prosthetic fractures. In addition, there is also the possibility of associating endomedullary pin elements such as locked Ender pins to the plate system, generating a mixed extramedullary and intramedullary fixation system, enhancing the advantages that both systems currently present separately.

Thus, the present invention discloses a cannulated fixation and locking set in the form of a “threaded pin head” which has a retractable area and when it is threaded into the hole of a plate, washer or other arrangement, with an angle that can be variable, it also generates a lock in its slide, determining the locking of whatever passes through its interior, be it wire, cable, thread, pin, etc. This determines a double locking.

In this way, locked Kirschners for osteosynthesis or tension-locked Kirschners are generated, a fundamental principle for the development of bone fixation systems with the Ilizarov concept (tension-locked Kirschners). Moreover, wire, cable or cerclage wire can be locked after tensioning without any problem. Also, mixed osteosynthesis systems (internal endomedullary and external extramedullary osteosynthesis systems) can be generated by being able to lock endomedullary interlocking elements (e.g. Ender pin) associated with a plate. In turn, extramedullary internal fixation systems can be generated with the concepts of Ilizarov, Kirschner under tension (Internal Ilizarov), being able to perform bone lengthening and angular corrections with internal osteosynthesis systems directed by magnetic systems such as those currently used by intramedullary lengthening systems.

Thus, by means of the invention, possible internal but extramedullary lengthening systems can be proposed, with the following advantages: Offering lengthening by internal systems to patients who, due to the bone size in length or the size of their endomedullary canal, cannot receive today an elongation treatment with internal pins. Possibility of entering the lengthening system without the need for a joint or physical approach. Minimizing bone damage by not requiring reaming of the medullary canal. And, being able to develop elements that allow internal angular corrections in addition to the lengthening that current endomedullary pins cannot treat, among many other advantages.

1. A cannulated locking and fixation set for orthopedic surgery comprising:
at least one internal fixation element comprising an external threaded tapered section, with an upper and lower surface interconnected by an internal through channel, at least one cross section being provided in said threaded section up to said lower surface, and
at least one external fixation element with at least one tapered hole with at least one threaded part that receives the internal fixation element.

2. The cannulated locking and fixation set in accordance with claim 1, wherein the upper side of the internal fixation element is machined to fit an adjustment tool.

3. The cannulated locking and fixation set in accordance with claim 1, wherein said internal fixation element is operationally located between the opening and closing positions by means of said adjustment tool.

4. The cannulated locking and fixation set in accordance with claim 1, wherein a bone fixation element passes through the internal fixation element and is operationally retained when the internal fixation element is in the closed position.

5. The cannulated locking and fixation set according to claim 4, wherein said bone fixation element is selected from the group consisting of Cerclage wires, pins with ogives, or Kirschner wires, pins, or a combination thereof.

6. The cannulated locking and fixation set in accordance with claim 1, wherein said external fixation element comprises a plurality of holes, each of which has a hole divided into a threaded portion and a smooth portion, both holes comprising a conical development.

7. The cannulated locking and fixation set in accordance with claim 1, wherein said external fixation element is selected from the group consisting of osteosynthesis plates, magnetic or electrical extension lock plates, Ilizarov rings, fixation thimbles, fixation spheres, or a combination thereof.

8. The cannulated locking and fixation set in accordance with claim 7, wherein said external fixation element is a fixation thimble which, together with the internal cannulated fixation element, forms a nose at the end of a Kirshner pin.

9. The cannulated locking and fixation set in accordance with claim 7, wherein said external fixation element is at least part of an Ilizarov ring.
